(19) United States
(12) Patent Application Publication
(10) Pub. No.: US 2022/0409672 A1
(43) Pub. Date: Dec. 29, 2022

(54) COMPOSITION COMPRISING GRYLLUS BIMACULATUS AS ACTIVE INGREDIENT FOR PREVENTION, ALLEVIATION, OR TREATMENT OF MUSCLE ATROPHY

(71) Applicant: Korea University Research and Business Foundation, Seoul (KR)

(72) Inventors: Jung Soon HAN, Seoul (KR); Si-Hyun KIM, Seoul (KR)

(73) Assignee: Korea University Research and Business Foundation, Seoul (KR)

(21) Appl. No.: 17/779,710

(22) PCT Filed: Jul. 6, 2021
(86) PCT No.: PCT/KR2021/008570

§ 371 Date: May 25, 2022

(30) Foreign Application Priority Data
Jul. 31, 2020 (KR) 10-2020-0096236

(51) Int. Cl. A61K 35/64; A61P 21/06

(52) U.S. Cl. CPC A61K 35/64; A61P 21/06

(57) ABSTRACT

The present invention relates to a composition for preventing, improving or treating muscle atrophy, which includes Gryllus bimaculatus, and more particularly, a Gryllus bimaculatus powder or Gryllus bimaculatus protein extract, as an active ingredient, and a method of preparing the same. The composition according to the present invention is effective in inhibiting the expression of muscle atrophy-associated key transcription factors, such as FoxO3 and MuRF-1, and is expected to be effectively used in muscle atrophy, various diseases caused thereby, muscle aging, undernourished patients, growing children and the elderly.

### TECHNICAL FIELD

The present invention relates to a composition for preventing, alleviating or treating muscle atrophy, which comprises Gryllus bimaculatus, more particularly, Gryllus bimaculatus powder or a protein extract of Gryllus bimaculatus powder, as an active ingredient.

This application claims priority to and the benefit of Korean Patent Application No. 10-2020-0096236, filed on Jul. 31, 2020, the disclosure of which is incorporated herein by reference in its entirety.

### BACKGROUND ART

According to the Food and Agriculture Organization (FAO), by 2050, it is expected that food resources need to at least double compared to 2010, so that there are serious concerns about the shortage of not only plant resources used as energy resources such as rice, barley and wheat, but also livestock, fish and shellfish and poultry that are edible as protein sources. Particularly, the shortage of animal protein sources is pointed out as the biggest problem. To solve the shortage of animal protein sources, the FAO has suggested a variety of edible insects that have been eaten all over the world for a long time in consideration of nutritional, environmental and economic aspects.

Edible insects have some differences in nutrient content depending on a species and a habitat, but generally contain 50 to 60% crude protein, 8.1 to 59% crude fat, 4.9 to 12.1% crude fiber, various inorganic components such as iron (Fe), calcium (Ca) and zinc (Zn) and vitamins, so the edible insects are highly available as food resources. In addition, edible insects do not have a high carbohydrate content, which is replaced by glucosamines, and are rich in unsaturated fatty acids and antioxidants such as tocopherol compared to general animal source foods. Moreover, edible insects are food materials that are expected to be used for the prevention and treatment of various metabolic diseases, especially, skeletal diseases, since almost no cholesterol is contained and a material such as chitin is contained.

Edible insects have been studied and industrialized in countries all over the world since the FAO announced a plan for revitalizing them as a future food resource in 2013, and in Korea, Gryllus bimaculatus was officially registered as a food raw material recognized by the Ministry of Food and Drug Safety in 2016.

Meanwhile, muscle atrophy is caused by lack of physical activity, bed rest due to injury, diabetes or aging, and refers to a symptom that results in a decrease in the size and number of muscle cells and a decrease in skeletal muscle mass over time, as well as a decrease in protein content and the loss of muscle strength. With the rapid increase of the elderly population worldwide, the risk of a falling accident in the elderly due to senile atrophy is continuously increasing, and most members of society also frequently suffer from atrophy caused by hypoactivity because of a decrease in physical activity. Particularly, it is known that a low intake of protein in the elderly, caused by a decline in digestive function, a decrease in proteases, a decrease in gastric acid secretion, a decrease in appetite, and problems with dental function, causes a decrease in synthesis of muscle protein. Such muscle atrophy is known to increase the risk of inflammations and infections that can cause various complications and physical problems, increase the risk of osteoporosis, and reduce the quality of life by inducing the expression of various skeletal diseases. To date, Gryllus bimaculatus research has only been conducted on breeding conditions, general ingredients, a feeding amount according to breeding density and fasting conditions according to food use development, and there is no study on the efficacy of Gryllus bimaculatus in relation to muscle atrophy.

### DISCLOSURE

### Technical Problem

The inventors found that Gryllus bimaculatus has an effect of reducing the expression of FoxO3 and MuRF-1, which are key transcription factors associated with muscle atrophy, and experimentally confirmed that muscle atrophy can be prevented, improved and treated by using Gryllus bimaculatus, and thus the present invention was completed.

Therefore, the present invention is directed to providing a food composition for preventing or alleviating muscle atrophy, which comprises Gryllus bimaculatus as an active ingredient.

The present invention is also directed to providing a health functional food composition for preventing or alleviating muscle atrophy, which comprises Gryllus bimaculatus as an active ingredient.

The present invention is also directed to providing a pharmaceutical composition for preventing or treating muscle atrophy, which comprises Gryllus bimaculatus as an active ingredient.

The present invention is also directed to providing a method of preparing a protein extract of Gryllus bimaculatus for preventing or alleviating muscle atrophy.

However, technical problems to be solved in the present invention are not limited to the above-described problems, and other problems which are not described herein will be fully understood by those of ordinary skill in the art from the following descriptions.

### Technical Solution

To achieve the purpose of the present invention, the present invention provides a food composition for preventing or alleviating muscle atrophy, which comprises Gryllus bimaculatus as an active ingredient.

In addition, the present invention provides a health functional food composition for preventing or alleviating muscle atrophy, which comprises Gryllus bimaculatus as an active ingredient.

In addition, the present invention provides a pharmaceutical composition for preventing or treating muscle atrophy, which comprises Gryllus bimaculatus as an active ingredient.

Furthermore, the present invention provides a method of preventing or treating muscle atrophy, which comprises administering the composition to a subject in need thereof.

In addition, the present invention provides a use of a pharmaceutical composition comprising Gryllus bimaculatus as an active ingredient for preventing, alleviating or treating muscle atrophy.

Further, the present invention provides a use of Gryllus bimaculatus for producing a drug used in prevention or treatment of muscle atrophy.

In one embodiment of the present invention, the Gryllus bimaculatus may be Gryllus bimaculatus powder.

In another embodiment of the present invention, the Gryllus bimaculatus powder may be prepared by steaming Gryllus bimaculatus at 100 to 150° C. for 40 to 60 minutes and powdering the resultant, but the present invention is not limited thereto.

In still another embodiment of the present invention, the Gryllus bimaculatus may be a Gryllus bimaculatus protein extract.

In yet another embodiment of the present invention, the Gryllus bimaculatus protein extract may be defatted.

In another embodiment of the present invention, the Gryllus bimaculatus protein extract may be extracted with one or more solvents selected from the group consisting of sodium phosphate solutions containing sodium hydroxide and sodium chloride, but the present invention is not limited thereto.

In still another embodiment of the present invention, the Gryllus bimaculatus protein extract may be extracted at 30 to 50° C. for 30 to 120 minutes, but the present invention is not limited thereto.

In yet another embodiment of the present invention, the Gryllus bimaculatus protein extract may be extracted by adding 0.1 to 0.5 M sodium hydroxide in a weight ratio of 1:10 to 20 (Gryllus bimaculatus powder:sodium hydroxide), but the present invention is not limited thereto.

In yet another embodiment of the present invention, the composition may reduce the expression of a Forkhead box protein O3 (FoxO3) or Muscle RING-finger protein-1 (MuRF-1) gene.

In yet another embodiment of the present invention, the Gryllus bimaculatus may be included at 0.1 μg/ml to 10 μg/ml with respect to the total composition, but the present invention is not limited thereto.

In yet another embodiment of the present invention, the muscle atrophy may be muscle atrophy due to the loss of muscle tissue caused by not using the muscle, muscle atrophy caused by a disease of the muscle itself, muscle atrophy caused by damage to a nerve that controls the muscle or aging-related muscle atrophy, but the present invention is not limited thereto.

In yet another embodiment of the present invention, the muscle atrophy may be one or more selected from the group consisting of aging-related muscle atrophy, degenerative muscle atrophy, diabetic muscle atrophy, limb-girdle type muscular dystrophy, Becker muscular dystrophy, Duchenne muscular dystrophy, myotonic muscular dystrophy, Emery-Dreifuss muscular dystrophy, distal muscular dystrophy, facioscapulohumeral muscular dystrophy, congenital muscular dystrophy and sarcopenic obesity, but the present invention is not limited thereto.

In addition, the present invention provides a method of preparing a Gryllus bimaculatus protein extract for preventing or alleviating muscle atrophy, which comprises: (a) powdering Gryllus bimaculatus; (b) defatting the powdered Gryllus bimaculatus; and (c) extracting a protein from the defatted Gryllus bimaculatus powder.

In one embodiment of the present invention, the preparation method may further comprise, before step (a), (a-1) fasting Gryllus bimaculatus; and (a-2) steaming the fasted Gryllus bimaculatus.

In another embodiment of the present invention, the fasting in step (a-1) may be performed for 24 to 72 hours, but the present invention is not limited thereto.

In another embodiment of the present invention, the steaming in step (a-2) may be performed at 100 to 150° C. for 40 to 60 minutes, but the present invention is not limited thereto.

In still another embodiment of the present invention, the defatting in step (b) may be performed using one or more solvents selected from the group consisting of ethanol, hexane and petroleum ether, but the present invention is not limited thereto.

In yet another embodiment of the present invention, the extraction in step (c) may be performed with one or more solvents selected from the group consisting of sodium phosphate solutions containing sodium hydroxide and sodium chloride, but the present invention is not limited thereto.

In yet another embodiment of the present invention, the preparation method may further comprise (d) desalting a Gryllus bimaculatus protein extract.

### Advantageous Effects

According to the present invention, Gryllus bimaculatus is effective in inhibiting the expression of muscle atrophy-associated key transcription factors, such as FoxO3 and MuRF-1. Accordingly, it may contribute to preventing senile diseases such as senile muscle atrophy, which is currently rapidly increasing and diseases caused thereby, aging and malnutrition, osteoporosis and fractures, as well as help prevent and treat serious muscle atrophy developed in patients in bed or by cancer. In addition, it is helpful in the prevention and treatment of various diseases caused by sarcopenic obesity.

### DESCRIPTION OF DRAWINGS

FIGS. 1A and 1B show results of analyzing cell viability per concentration of an extract using MTT analysis, in which FIG. 1A is a result of treating Gryllus bimaculatus powder dissolved in DMSO, and FIG. 1B is a result of treating a Gryllus bimaculatus protein extract dissolved in DMSO.

FIG. 2 shows a result of measuring mRNA expression levels of FoxO3 (left graph) and MuRF-1 (right graph) by treating musculoskeletal cells with a Gryllus bimaculatus powder dissolved in DMSO by concentration.

FIG. 3 is the flowchart illustrating a method of preparing a Gryllus bimaculatus powder and a Gryllus bimaculatus protein extract for preventing or alleviating muscle atrophy in time series.

### BEST MODE

Various causes for the mechanism of developing muscle atrophy of skeletal muscles have been suggested, and it is known that, among them, when a muscle RING-finger protein-1 (MuRF-1) factor known as a key transcription factor associated with muscle atrophy is expressed, muscle growth and differentiation are inhibited and the catabolism of skeletal muscle protein is activated, thereby causing muscle atrophy and various muscle-related diseases. It is reported that a Forkhead box protein 03 (Fox03) transcription factor, also a key transcription factor associated with muscle atrophy, is expressed during atrophy of skeletal muscle fibers, thereby inhibiting the proliferation and differentiation of satellite cells, and promotes protein degradation.

The inventors confirmed that Gryllus bimaculatus does not have toxicity to muscle cells (refer to Example 2), is effective in inhibiting the expression of FoxO3 and MuRF-1, which are muscle atrophy-associated transcription factors (refer to Example 3), and found that a composition comprising Gryllus bimaculatus as an active ingredient can prevent muscle atrophy and alleviate its symptoms, and also treat muscle atrophy.

Accordingly, the present invention can provide a food composition for preventing or alleviating muscle atrophy, which comprises Gryllus bimaculatus as an active ingredient.

In another aspect of the present invention, the present invention may provide a health functional food composition for preventing or alleviating muscle atrophy, which comprises Gryllus bimaculatus as an active ingredient.

In still another aspect of the present invention, the present invention may provide a pharmaceutical composition for preventing or treating muscle atrophy, which comprises Gryllus bimaculatus as an active ingredient.

The term “Gryllus bimaculatus” used herein, called “ssang-byeol-gwi-ttu-la-mi” or “ssang-byeol” in Korean, is an insect in the family Gryllidae and the order Orthoptera, registered as a food raw material in Korean, along with grasshoppers, edible pupae, while silkworms, Tenebrio molitor larvae (mealworms), white-spotted flower chafer beetle (Protaetia brevitarsis seulensis) larvae, and Allomyrina dichotoma larvae. The Gryllus bimaculatus according to the present invention may be collected from nature or commercially available.

The term “muscle atrophy or muscular atrophy” refers to any symptom in which a decrease in muscle tissue or muscle degeneration appears.

In the present invention, the Gryllus bimaculatus may be a Gryllus bimaculatus powder or a Gryllus bimaculatus protein extract.

In the present invention, the Gryllus bimaculatus powder may be prepared by steaming Gryllus bimaculatus at 100 to 150° C. for 40 to 60 minutes and powdering the resultant.

In the present invention, the Gryllus bimaculatus protein extract may be defatted, but the present invention is not limited thereto. As a defatting solvent, ethanol, hexane or petroleum ether may be used, and defatting may also be performed by other known methods, for example, compression extraction, etc.

In the present invention, the Gryllus bimaculatus protein extract may be extracted with one or more solvents selected from the group consisting of sodium phosphate solutions containing sodium hydroxide and sodium chloride, but the present invention is not limited thereto.

In the present invention, the Gryllus bimaculatus protein extract may be extracted at 30 to 50° C. for 30 to 120 minutes, and when sodium hydroxide is used as an extraction solvent, may be extracted by mixing 0.1 to 0.5M sodium hydroxide with Gryllus bimaculatus powder at a weight ratio of 1:10 to 20 (Gryllus bimaculatus powder:sodium hydroxide).

In the present invention, the food composition, the health functional food composition and the pharmaceutical composition have an effect of reducing the expression of a FoxO3 or MuRF-1 gene. Therefore, they have the effect of preventing, alleviating and treating muscle atrophy.

In the present invention, the Gryllus bimaculatus may be included at 0.1 μg/ml to 10 μg/ml with respect to the total composition according to the present invention. It was confirmed that, in the above concentration range, the Gryllus bimaculatus has no toxicity to musculoskeletal cells and significantly inhibits the expression of a muscle atrophy-associated key transcription factor.

In the present invention, the muscle atrophy may be muscle atrophy due to the loss of muscle tissue caused by not using the muscle, muscle atrophy caused by a disease of the muscle itself, muscle atrophy caused by damage to a nerve that controls the muscle or aging-related muscle atrophy, and more particularly, aging-related muscle atrophy, degenerative muscle atrophy, diabetic muscle atrophy, limb-girdle type muscular dystrophy, Becker muscular dystrophy, Duchenne muscular dystrophy, myotonic muscular dystrophy, Emery-Dreifuss muscular dystrophy, distal muscular dystrophy, facioscapulohumeral muscular dystrophy, congenital muscular dystrophy, or sarcopenic obesity, etc, but the present invention is not limited thereto.

Meanwhile, when Gryllus bimaculatus according to the present invention is used as a food additive, the Gryllus bimaculatus may be added in the form of a powder or protein extract or used in combination with a food or food ingredient, and may be suitably used according to a conventional method. The mixing amount of the active ingredient may be appropriately determined according to the purpose of use (prevention, health or therapeutic treatment). In general, in production of foods or beverages, the Gryllus bimaculatus of the present invention may be added at 20 to 40 wt % with respect to the raw material.

However, in the case of long-term intake for health and hygiene or health control, the above-described amount may be less than the above range, and since there is no problem in safety, the active ingredient may be used in an amount above the above range.

There is no particular limitation on the type of food. Examples of the food to which the above-described material can be added include meat, sausage, bread, chocolate, candy, snacks, confectioneries, pizza, ramen, other noodles, gums, dairy products including ice cream, various soups, sources, jelly, energy bars, beverages, tea, drinks, alcoholic beverages, hangover relievers and vitamin complexes, and include all types of health functional foods in the common sense.

A health beverage composition according to the present invention may contain various flavoring agents or natural carbohydrates as additional ingredients, like common beverages. The above-described natural carbohydrates are monosaccharides such as glucose, fructose, etc.; disaccharides such as maltose, sucrose, etc.; and polysaccharides such as dextrin, cyclodextrin, etc., and sugar alcohols such as xylitol, sorbitol, erythritol, etc. As a sweetening agent, a natural sweetener such as thaumatin or a stevia extract, or a synthetic sweetener such as saccharin or aspartame, may be used. Generally, the proportion of the natural carbohydrate is approximately 0.01 to 0.20 g, or approximately 0.04 to 0.10 g per 100 mL of the composition of the present invention.

In addition to the above ingredients, the composition according to the present invention may contain various nutrients, vitamins, minerals, flavoring agents, coloring agents, pectic acid and a salt thereof, alginic acid and a salt thereof, an organic acid, protective colloid thickening agents, pH adjusters, stabilizers, preservatives, glycerin, alcohols, or carbonating agents used in carbonated beverages. In addition, the composition of the present invention may contain flesh for preparing natural fruit juices, fruit juice drinks and vegetable juices. Such an ingredient may be used independently or in combination. The proportion of this additive is not critical, but is generally selected in the range of 0.01 to 0.20 parts by weight per 100 parts by weight of the composition of the present invention.

The term “health functional food” used herein refers to foods produced and processed using a raw material or ingredient with functionality useful for the human body according to Health Functional Food Act No. 6727, and foods eaten for the purpose of obtaining a useful effect for health care such as nutrient regulation for the structure and function of the human body or physiological actions. The health functional food of the present invention may include a conventional food additive, and unless specified otherwise, suitability as a food additive is determined according to the specifications and standards for a corresponding item in accordance with the general rules and general test methods of the Korean Food Additives Codex approved by the Ministry of Food and Drug Safety.

Meanwhile, the pharmaceutical composition for preventing or treating muscle atrophy comprising the Gryllus bimaculatus according to the present invention as an active ingredient may further comprise a suitable carrier, excipient and diluent, which are conventionally used in the preparation of the pharmaceutical composition. The excipient may be one or more selected from the group consisting of a diluent, a binder, a disintegrant, a lubricant, an adsorbent, a humectant, a film-coating material and a controlled-release additive.

The pharmaceutical composition according to the present invention may be formulated in the form of a powder, a granule, a sustained-release granule, an enteric granule, a solution and a liquid, an ophthalmic solution, an elixir, an emulsion, a suspension, a spirit, a troche, aromatic water, a lemonade, a tablet, a sustained-release tablet, an enteric tablet, a sublingual tablet, a hard capsule, a soft capsule, a sustained-release capsule, an enteric capsule, a pill, a tincture, a soft extract, a dry extract, a fluid extract, an injection, a capsule, a perfusate, a plaster, a lotion, a paste, a spray, an inhalant, a patch, a sterile injection, or an external preparation such as an aerosol according to a conventional method, and the external preparation may be formulated in a cream, a gel, a patch, a spray, an ointment, a plaster, a lotion, a liniment, a paste or a cataplasma.

The carrier, excipient and diluent that may be included in the pharmaceutical composition according to the present invention may include lactose, dextrose, sucrose, an oligosaccharide, sorbitol, mannitol, xylitol, erythritol, maltitol, starch, acacia gum, alginate, gelatin, calcium phosphate, calcium silicate, cellulose, methyl cellulose, microcrystalline cellulose, polyvinyl pyrrolidone, water, methylhydroxybenzoate, propylhydroxybenzoate, talc, magnesium stearate and mineral oil.

The pharmaceutical composition of the present invention may be formulated with a diluent or an excipient such as a filler, a thickening agent, a binder, a wetting agent, a disintegrant or a surfactant, which is generally used.

As additives for a tablet, powder, granule, capsule, pill and troche, excipients such as corn starch, potato starch, wheat starch, lactose, sucrose, glucose, fructose, di-mannitol, precipitated calcium carbonate, synthetic aluminum silicate, calcium monohydrogen phosphate, calcium sulfate, sodium chloride, sodium bicarbonate, purified lanolin, microcrystalline cellulose, dextrin, sodium alginate, methylcellulose, sodium carboxymethylcellulose, kaolin, urea, colloidal silica gel, hydroxypropyl starch, hydroxypropyl methyl cellulose (HPMC), HPMC 1928, HPMC 2208, HPMC 2906, HPMC 2910, propylene glycol, casein, calcium lactate and Primojel; binders such as gelatin, gum arabic, ethanol, agar powder, cellulose acetate phthalate, carboxymethyl cellulose, carboxymethyl cellulose calcium, glucose, purified water, sodium caseinate, glycerin, stearic acid, sodium carboxymethylcellulose, sodium methylcellulose, methylcellulose, microcrystalline cellulose, dextrin, hydroxycellulose, hydroxypropyl starch, hydroxymethylcellulose, purified shellac, starch powder, hydroxypropylcellulose, hydroxypropylmethylcellulose, polyvinyl alcohol and polyvinylpyrrolidone; disintegrants such as hydroxypropylmethylcellulose, corn starch, agar powder, methylcellulose, bentonite, hydroxypropyl starch, sodium carboxymethylcellulose, sodium alginate, carboxymethyl cellulose calcium, calcium citrate, sodium lauryl sulfate, silicic anhydride, 1-hydroxypropyl cellulose, dextran, an ion exchange resin, polyvinyl acetate, formaldehyde-treated casein and gelatin, alginic acid, amylose, guar gum, sodium bicarbonate, polyvinylpyrrolidone, calcium phosphate, gelled starch, gum arabic, amylopectin, pectin, sodium polyphosphate, ethyl cellulose, sucrose, magnesium aluminum silicate, a di-sorbitol solution and light anhydrous silicic acid; and lubricants such as calcium stearate, magnesium stearate, stearic acid, hydrogenated vegetable oil, talc, limestone, kaolin, petrolatum, sodium stearate, cacao butter, sodium salicylate, magnesium salicylate, polyethylene glycol (PEG) 4000 and PEG 6000, liquid paraffin, hydrogenated soybean oil (Lubri wax), aluminum stearate, zinc stearate, sodium lauryl sulfate, magnesium oxide, Macrogol, synthetic aluminum silicate, silicic anhydride, a higher fatty acid, a higher alcohol, silicone oil, paraffin oil, polyethylene glycol fatty acid ether, starch, sodium chloride, sodium acetate, sodium oleate, dileucine and light anhydrous silicic acid may be used.

Additives for a liquid according to the present invention may be water, diluted hydrochloric acid, diluted sulfuric acid, sodium citrate, monostearate sucrose, polyoxyethylene sorbitol fatty acid esters (Tween esters), polyoxyethylene monoalkylethers, lanolin ethers, lanolin esters, acetic acid, hydrochloric acid, acetic acid, hydrochloric acid, aqueous ammonia, ammonium carbonate, potassium hydroxide, sodium hydroxide, prolamine, polyvinylpyrrolidone, ethyl cellulose, and sodium carboxymethylcellulose.

For a syrup according to the present invention, a sucrose solution, another type of sugar or a sweetener may be used, and a flavoring agent, a coloring agent, a preservative, a stabilizer, a suspending agent, an emulsifier or a thickener may be used as needed.

For an emulsion according to the present invention, an emulsifier, a preservative, a stabilizer or a flavoring agent may be used as needed.

For a suspension according to the present invention, a suspending agent such as acacia, tragacanth, methylcellulose, carboxymethylcellulose, sodium carboxymethylcellulose, microcrystalline cellulose, sodium alginate, hydroxypropylmethylcellulose, HPMC 1828, HPMC 2906 or HPMC 2910 may be used, and a surfactant, a preservative, a stabilizer, a coloring agent, a fragrance may be used as needed.

For an injection according to the present invention, a solvent such as injectable sterile water, 0.9% sodium chloride for injection, Ringer's solution, dextrose for injection, dextrose+sodium chloride for injection, PEG, lactated Ringer's solution, ethanol, propylene glycol, non-volatile oil-sesame oil, cottonseed oil, peanut oil, soybean oil, corn oil, ethyl oleate, isopropyl myristic acid or benzene benzoate; a solubilizing agent such as sodium benzoate, sodium salicylate, sodium acetate, urea, urethane, monoethylacetamine, butazolidine, propylene glycol, Tween, nicotinamide, hexamine or dimethylacetamide; a buffer such as a weak acid and a salt thereof (acetic acid and sodium acetate), a weak base and a salt thereof (ammonia and ammonium acetate), an organic compound, a protein, albumin, peptone, or gums; an isotonic agent such as sodium chloride; a stabilizer such as sodium bisulfite (NaHSO₃), carbon dioxide gas, sodium metabisulfite (Na₂S₂O₅), sodium sulfite (Na₂SO₃), nitrogen gas (N₂) or ethylenediaminetetracetic acid; an sulfation agent such as sodium bisulfide 0.1%, sodium formaldehyde sulfoxylate, thiourea, disodium ethylenediaminetetraacetate or acetone sodium bisulfite; a pain-relief agent such as benzyl alcohol, chlorobutanol, procaine hydrochloride, glucose or calcium gluconate; or a suspending agent such as sodium CMC, sodium alginate, Tween 80 or aluminum monostearate may be used.

For a suppository according to the present invention, a base such as cacao butter, lanolin, Witepsol, polyethylene glycol, glycerogelatin, methyl cellulose, carboxymethylcellulose, a mixture of stearate and oleate, Subanal, cottonseed oil, peanut oil, palm oil, cacao butter+cholesterol, lecithin, Lanette wax, glycerol monostearate, Tween or Span, Imhausen, monolene (propylene glycol monostearate), glycerin, Adeps solidus, Buytyrum Tego-G, Cebes Pharma 16, hexalide base 95, Cotomar, Hydrokote SP, S-70-XXA, S-70-XX75 (S-70-XX95), Hydrokote 25, Hydrokote 711, Idropostal, Massa estrarium, A, AS, B, C, D, E, I, T), Mass-MF, Masupol, Masupol-15, neosuppostal-N, paramount-B, supposiro (OSI, OSIX, A, B, C, D, H, L), suppository base IV types (AB, B, A, BC, BBG, E, BGF, C, D, 299), Suppostal (N, Es), Wecoby (W, R, S, M, Fs), or a Tegester triglyeride base (TG-95, MA, 57) may be used.

A solid formulation for oral administration may be a tablet, a pill, a powder, a granule or a capsule, and such a solid formulation may be prepared by mixing at least one of excipients, for example, starch, calcium carbonate, sucrose, lactose and gelatin, with the active ingredient. Also, in addition to the simple excipient, lubricants such as magnesium stearate and talc may also be used.

As a liquid formulation for oral administration, a suspension, a liquid for internal use, an emulsion, or a syrup may be used, and a generally-used simple diluent such as water or liquid paraffin, as well as various types of excipients, for example, a wetting agent, a sweetener, a fragrance and a preservative may be included. A formulation for parenteral administration may be a sterilized aqueous solution, a non-aqueous solvent, a suspension, an emulsion, a lyophilizing agent or a suppository. As the non-aqueous solvent or suspension, propylene glycol, polyethylene glycol, a vegetable oil such as olive oil, or an injectable ester such as ethyl oleate may be used.

The composition of the present invention may be administered to a subject in need by various routes. For example, the composition of the present invention may be administered through oral administration, intranasal administration, transbronchial administration, cervical administration, arterial injection, central intravenous injection, peripheral intravenous injection, subcutaneous injection, intramuscular injection or intraperitoneal injection, gastrostomy, or enterostomy. A daily dose may be administered once or in multiple divided portions.

In yet another aspect of the present invention, the present invention may provide a method of preparing a Gryllus bimaculatus protein extract for preventing or alleviating muscle atrophy, which comprises: (a) powdering Gryllus bimaculatus into a powder; (b) defatting the powdered Gryllus bimaculatus; and (c) extracting a protein from the defatted Gryllus bimaculatus powder.

In the present invention, the preparation method may further comprise, before step (a), (a-1) fasting Gryllus bimaculatus; and (a-2) steaming the fasted Gryllus bimaculatus.

In the present invention, the fasting in step (a-1) may be performed for 24 to 72 hours. The Gryllus bimaculatus is grown while suitable temperature and humidity are maintained, and is mainly grown on a diet of sawdust or wheat bran. When Gryllus bimaculatus was processed without fasting, due to residual feed and excrement in the intestines of Gryllus bimaculatus, preference was reduced by a strong taste and odor although Gryllus bimaculatus was processed. However, the contents of feed and excrement in the intestines may be lowered by fasting Gryllus bimaculatus. More specifically, the fasting may be performed for 24 to 72 hours. When the fasting is performed for less than 1 hour, the feed and excrement in the intestines of Gryllus bimaculatus are not sufficiently removed, so that bad taste and odor may remain after processing, and when the fasting is performed for more than 72 hours, there is a problem in that the growth of Gryllus bimaculatus is inhibited. In addition, before the fasting step, the bad taste and odor of Gryllus bimaculatus may be further reduced by growth with a flour diet.

In the present invention, the steaming in step (a-2) may be performed at 100 to 150° C. for 40 to 60 minutes.

In the present invention, the defatting in step (b) may be performed using one or more solvents selected from the group consisting of ethanol, hexane and petroleum ether, but the present invention is not limited thereto.

In the present invention, the extraction in step (c) may be performed with one or more solvents selected from the group consisting of sodium phosphate solutions containing sodium hydroxide and sodium chloride, but the present invention is not limited thereto. A supernatant was obtained by centrifugation after sufficiently mixing the solvent and the defatted Gryllus bimaculatus powder obtained in step (b), and then centrifuged again to collect a pellet thereof, thereby obtaining a protein extract.

In addition, in the present invention, the preparation method may further comprise (d) desalting a Gryllus bimaculatus protein extract. The desalting may be performed by a known method, and in one embodiment of the present invention, the desalting may be performed using a dialysis bag (12 kDa MWCO) for 12 hours.

Terms and words used in the specification and claims should not be construed as being limited to general or dictionary terms meanings, and should be interpreted with the meaning and concept in accordance with the technical idea of the present invention based on the principle that the inventors have appropriately defined the concepts of terms in order to explain the invention in the best way.

### MODES OF THE INVENTION

Hereinafter, to help in understanding the present invention, exemplary examples will be suggested. However, the following examples are merely provided to more easily understand the present invention, and not to limit the present invention.

### EXAMPLES

### Example 1. Preparation of Gryllus bimaculatus Powder and Protein Extract

1.1. Gryllus Bimaculatus Powder

A Gryllus bimaculatus powder was purchased from a cricket farm (Chungbuk, Korea). The powder was prepared by washing Gryllus bimaculatus that had been fasted for 2 days, steaming it for 40 to 60 minutes, drying the steamed result at 60° C. for approximately 18 hours, and grinding the dried result into a powder. The purchased Gryllus bimaculatus powder was freeze-dried at −24° C. or less for use.

1.2. Defatting of Gryllus bimaculatus Powder

To increase protein extraction efficiency, a defatting process was performed on the Gryllus bimaculatus powder. More specifically, 99.5% ethanol was added to the Gryllus bimaculatus powder at a ratio of 1:5 for extraction in a shaking bath (VS-1205SW1, Vision Scientific Co., Ltd., Korea) at 40° C. for 60 minutes. This process was repeated twice, and then ethanol was evaporated and dried for 12 hours.

1.3. Process of Extracting Gryllus bimaculatus Protein

To the defatted Gryllus bimaculatus powder obtained in Example 1.2, 0.25M sodium hydroxide (NaOH) was added in a ratio of 1:15, and mixed using a hot plate (Vision Scientific Co., Ltd., Korea) and a magnetic stirrer at 40° C. for 60 minutes. Afterward, the resulting mixture was centrifuged using a centrifuge (VS-24SMTi, Vision Science Co., Ltd., Korea) at 4° C. and 3,000 rpm for 10 minutes, followed by collecting only a supernatant. The obtained supernatant was adjusted to pH 4.4, and centrifuged again at 4° C. and 3,500 rpm for 10 minutes, thereby obtaining a pellet.

1.4. Dialysis and Freeze-Drying of Pellet

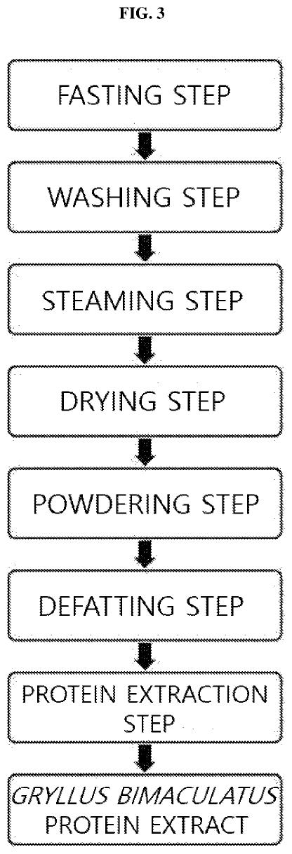

The pellet obtained in Example 1.3 was input into a dialysis bag (12 kDa MWCO; Sigma-Aldrich Chemical Co., U.S.A.) for desalting for 12 hours. Afterward, a protein extract of Gryllus bimaculatus was obtained by freeze-drying for 36 hours. The obtained protein extract was stored at −24° C. for an additional experiment. A method of preparing the Gryllus bimaculatus protein extract is illustrated in the flowchart of FIG. 3.

1.5. Preparation of Composition Including Gryllus bimaculatus for In Vitro Experiment

10 mg of the Gryllus bimaculatus powder or Gryllus bimaculatus protein extract obtained in Example 1 was added to 1 ml of dimethyl sulfoxide (DMSO), and dissolved using an ultrasonic homogenizer (Sonicator Vibra cell, Sonics & Materials; amplitude adjustment 40%). After centrifugation at 13,000 rpm and 10° C. for 10 minutes, only a supernatant was collected, and then transferred to a 1 ml Eppendorf tube (e-tube; micro centrifuge tube).

### Example 2. Evaluation of Cytotoxicity of Gryllus bimaculatus

First, to evaluate the toxicity of the Gryllus bimaculatus powder or Gryllus bimaculatus protein extract according to the present invention to musculoskeletal cells, which are C2C12 cells, 3-(4,5-dimethylthiazol-2-yl)-2,5-diphenyltetrazolium bromide (MTT) analysis was performed. The toxicity is measured by using a principle of reducing tetrazolium into blue-violet non-aqueous MTT formazan crystal by breaking the ring structure of the tetrazolium by a dehydrogenase present in the mitochondria of living cells (with intact metabolism) when the cells were treated with yellow aqueous MTT tetrazolium. The MTT formazan crystal is generally dissolved in an organic solvent such as dimethyl sulfoxide to measure absorbance using an ELISA reader. Here, the absorbance of the formazan dye was detected at approximately 540 nm, indicating an amount of MTT reduced by living cells and proportional to the number of viable cells present in each well.

The mouse musculoskeletal cells (C2C12 cells) were treated by concentration, for example, 1 mg/mL, 0.1 mg/mL and 0.01 mg/mL, with the Gryllus bimaculatus powder or Gryllus bimaculatus protein extract dissolved in DMSO prepared in Example 1.5 for 24 hours, treated with an MTT reagent, followed by a reaction at 37° C. in an incubator for 2 hours. Subsequently, the MTT reagent was removed, DMSO was added to completely dissolve formazan generated in the wells and then 200 μl was aliquoted into each well of a 96-well plate. Absorbance was measured at 540 nm using an ELISA reader (microplate spectrophotometer, Epoch, BioTek), and cell viability was calculated.

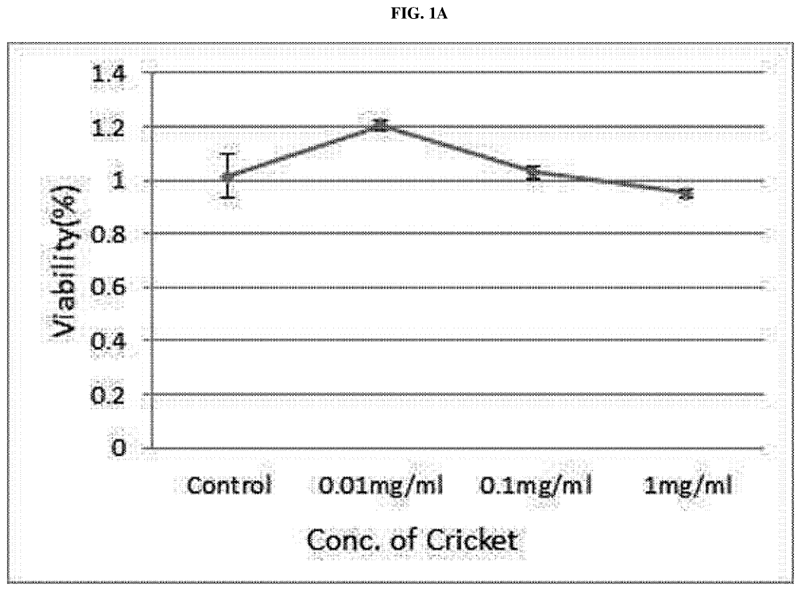

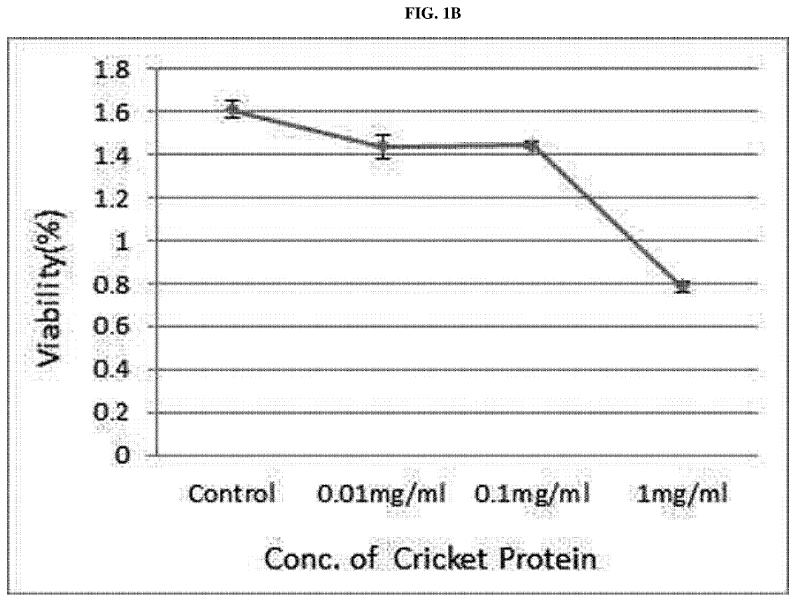

As a result, referring to FIGS. 1A and 1B, it was confirmed that both of the Gryllus bimaculatus powder (FIG. 1A) and the Gryllus bimaculatus protein extract (FIG. 1B) have no toxicity to musculoskeletal cells (C2C12 cells). Accordingly, the inventors observed a change in the expression of a target transcription factor in musculoskeletal cells according to treatment with the Gryllus bimaculatus powder or Gryllus bimaculatus protein extract.

### Example 3. Confirmation of Effect of Inhibiting Expression of Muscle Atrophy-Associated Transcription Factors, FoxO3 and MuRF-1, of Gryllus bimaculatus

To examine whether Gryllus bimaculatus is effective in preventing, alleviating or treating muscle atrophy, mRNA levels of FoxO, which is a muscle atrophy-associated key transcription factor, and MuRF-1, which is a muscle proteolysis gene, were comparatively analyzed. First, total RNA of cells prepared under the same conditions was isolated and quantified using a TRIzol reagent (Invitrogen Co., U.S.A.), and primers, diethyl pyrocarbonate (DEPC) water and a one-step RT PCR premix kit (Intron, Korea) were added, and then amplified DNA was detected using a qRT-PCR (quantitative real time polymerase chain reaction; Applied Biosystems, QuantStudio 3) apparatus.

More specifically, the concentration of Gryllus bimaculatus to be injected was confirmed through an MTT experiment of Example 2, and then C2C12 cells were incubated at 37° C. in an incubator for 12 hours or more. A medium was removed from the C2C12 cells incubated for 12 hours or more, and then 1 mL of a medium was injected. Subsequently, 1 μl of the sample was added to each well (PBS treatment in the control) and mixed, followed by incubation at 37° C. in an incubator. After 12 hours, the medium was removed, and then the cells were washed twice with PBS. After washing, 200 μl of a TRIzol reagent was added to each well, left at room temperature for 5 minutes, and then mixed with 40 μl of chloroform. Afterward, following centrifugation at 13,200 rpm and 4° C. for 15 minutes, 120 μl of isopropanol was added to the supernatant, and left at room temperature for 10 minutes. After 10 minutes, centrifugation was performed at 13,200 rpm and 4° C. for 15 minutes, a supernatant was removed, 100 μl of 70% cold ethanol was injected, and then centrifugation was performed at 13,200 rpm and 4° C. for 15 minutes. Here, the process of injecting 70% cold ethanol and performing centrifugation was repeated twice. Afterward, the 70% ethanol was removed, DEPC water was added, and an RNA concentration was measured using a microplate spectrophotometer (Epoch, BioTek). After the RNA concentration of the sample was measured, accordingly, the sample and DEPC water were additionally added, and 2 μl of TOPreal™ qPCR 2× premix (SYBR Green with high ROX) was injected. The mixed sample was transferred to a zipperstrip PCR tube to amplify DNA using a PCR apparatus (Thermal Cycler; Bio-Rad). After amplification was completed, the resulting product was transferred to a microcentrifuge tube, and 2 μl of the sample, 5 μl of 2× master mix, 2.5 μl of pure water and 0.5 μl of a primer were injected into a PCR plate. Subsequently, each of mGAPDH and mMSTN was injected into one sample. Finally, the resulting sample was sealed, and amplified DNA was detected using a RT-PCR apparatus (Applied Biosystems, QuantStudio 3).

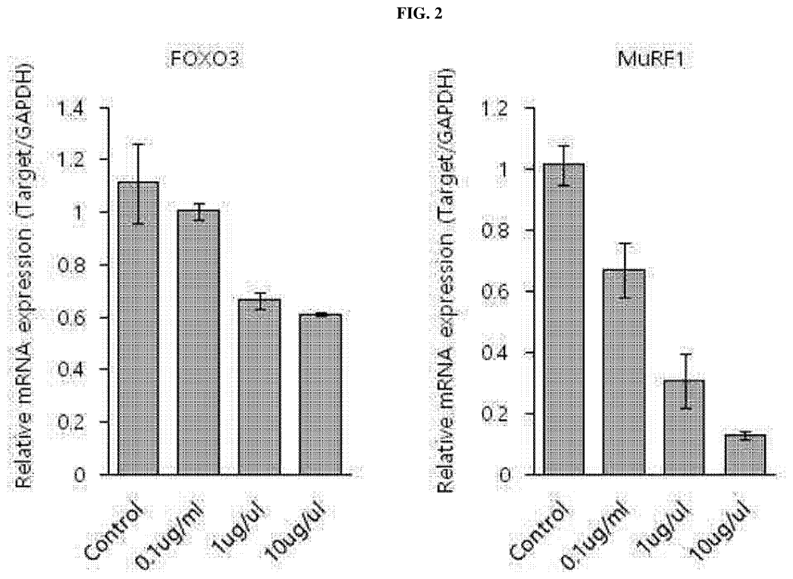

As a result, as shown in FIG. 2, it was confirmed that Gryllus bimaculatus, and more particularly, the Gryllus bimaculatus powder and the Gryllus bimaculatus protein extract are effective in reducing the expression of FoxO3 and MuRF-1 in a concentration-dependent manner. The FoxO transcription factor that is known as a key factor involved in skeletal muscle fiber atrophy and damage is expressed during the atrophy of skeletal muscle fibers, and is known to not only inhibit the proliferation and differentiation of satellite cells but also promote protein degradation (Kamei & Ogawa, 2003). That is, the above result shows that the Gryllus bimaculatus powder or Gryllus bimaculatus protein extract can play a positive role in the inhibition and prevention of muscle atrophy by inhibiting FoxO expression. In skeletal muscles, FoxO consists of three different forms, such as FoxO1, FoxO3 and FoxO4, and when FoxO expression increases, protein synthesis may be inhibited, and thus it is very important to inhibit FoxO expression. The inventors confirmed that, as shown in the left graph of FIG. 2, the Gryllus bimaculatus powder or Gryllus bimaculatus protein extract has an effect of significantly reducing FoxO3 expression in a concentration-dependent manner, confirming the possibility of preventing and treating muscle atrophy.

In addition, it is known that muscle atrophy is triggered by increasing the expression of a muscle proteolysis gene MuRF-1 (Cai et al., 2004), and as a result of also examining the expression of MuRF-1 expressed by FoxO in the nucleus of a skeletal muscle, as shown in the right graph of FIG. 2, it was confirmed that the Gryllus bimaculatus powder or Gryllus bimaculatus protein extract is effective in significantly reducing MuRF-1 expression in a concentration-dependent manner, confirming the possibility of inhibiting muscle atrophy.

The above findings prove that the Gryllus bimaculatus powder or Gryllus bimaculatus protein extract is effective in preventing muscle atrophy by inhibiting the occurrence thereof, and also can alleviate and treat the symptoms of muscle atrophy.

It should be understood by those of ordinary skill in the art that the above description of the present invention is exemplary, and the exemplary embodiments disclosed herein can be easily modified into other specific forms without departing from the technical spirit or essential features of the present invention. Therefore, the exemplary embodiments described above should be interpreted as illustrative in all aspects and not restrictive

### INDUSTRIAL APPLICABILITY

According to the present invention, Gryllus bimaculatus is effective in inhibiting the expression of muscle atrophy-associated key transcription factors, such as FoxO3 and MuRF-1. Accordingly, it may contribute to preventing senile diseases such as senile muscle atrophy, which is currently rapidly increasing and diseases caused thereby, aging and malnutrition, osteoporosis and fractures, as well as help prevent and treat serious muscle atrophy developed in patients in bed or by cancer. In addition, it is helpful in the prevention and treatment of various diseases caused by sarcopenic obesity, and thus the present invention has industrial applicability.

1. A method of treating or alleviating muscle atrophy, comprising administering to a subject in need thereof a composition comprising an effective amount of Gryllus bimaculatus.

2. The method of claim 1, wherein the Gryllus bimaculatus is a Gryllus bimaculatus powder.

3. The method of claim 2, wherein the Gryllus bimaculatus powder is prepared by steaming Gryllus bimaculatus at 100 to 150° C. for 40 to 60 minutes and powdering the resultant.

4. The method of claim 1, wherein the Gryllus bimaculatus is a Gryllus bimaculatus protein extract.

5. The method of claim 4, wherein the Gryllus bimaculatus protein extract is defatted.

6. The method of claim 4, wherein the Gryllus bimaculatus protein extract is extracted with one or more solvents selected from the group consisting of sodium phosphate solutions containing sodium hydroxide and sodium chloride.

7. The method of claim 4, wherein the Gryllus bimaculatus protein extract is extracted at 30 to 50° C. for 30 to 120 minutes.

8. The method of claim 4, wherein the Gryllus bimaculatus protein extract is extracted by adding 0.1 to 0.5M sodium hydroxide in a weight ratio of 1:10 to 20 (Gryllus bimaculatus powder:sodium hydroxide).

9. The method of claim 1, wherein the composition reduces the expression of Forkhead box protein O3 (FoxO3) or a muscle RING-finger protein-1 (MuRF-1) gene.

10. The method of claim 1, wherein the Gryllus bimaculatus is contained at 0.1 μg/ml to 10 μg/ml with respect to the total composition.

11. The method of claim 1, wherein the muscle atrophy is muscle atrophy due to the loss of muscle tissue caused by not using the muscle, muscle atrophy caused by a disease of the muscle itself, muscle atrophy caused by damage to a nerve that controls the muscle, or aging-related muscle atrophy.

12. The method of claim 1, wherein the muscle atrophy is one or more selected from the group consisting of aging-related muscle atrophy, degenerative muscle atrophy, diabetic muscle atrophy, limb-girdle type muscular dystrophy, Becker muscular dystrophy, Duchenne muscular dystrophy, myotonic muscular dystrophy, Emery-Dreifuss muscular dystrophy, distal muscular dystrophy, facioscapulohumeral muscular dystrophy, congenital muscular dystrophy and sarcopenic obesity.

13. The method of claim 1, wherein the composition is food composition, health functional food composition, or pharmaceutical composition.

14-16. (canceled)

17. A method of preparing a Gryllus bimaculatus protein extract for preventing or alleviating muscle atrophy, the method comprising:
(a) powdering Gryllus bimaculatus;
(b) defatting the powdered Gryllus bimaculatus; and
(c) extracting a protein from the defatted Gryllus bimaculatus powder.

18. The method of claim 17, further comprising:
prior to step (a),
(a-1) fasting Gryllus bimaculatus; and
(a-2) steaming the fasted Gryllus bimaculatus.

19. The method of claim 18, wherein the fasting in step (a-1) is performed for 24 to 72 hours.

20. The method of claim 18, wherein the steaming in step (a-2) is performed at 100 to 150° C. for 40 to 60 minutes.

21. The method of claim 17, wherein the defatting in step (b) is performed using one or more solvents selected from the group consisting of ethanol, hexane and petroleum ether.

22. The method of claim 17, wherein the extraction in step (c) is performed with one or more solvents selected from the group consisting of sodium phosphate solutions containing sodium hydroxide and sodium chloride.

23. The method of claim 17, further comprising:
(d) desalting a Gryllus bimaculatus protein extract.

24-26. (canceled)
